(19) United States
(12) Patent Application Publication
(10) Pub. No.: US 2023/0277836 A1
(43) Pub. Date: Sep. 7, 2023

(54) SENSOR DEVICE FOR SENSING AT LEAST ONE FUNCTIONAL VALUE OF A MEDICAL DEVICE AND A METHOD FOR OPERATING THE SENSOR DEVICE

(71) Applicant: Kardion GmbH, Stuttgart (DE)

(72) Inventors: Inga Schellenberg, Stuttgart (DE); Hardy Baumbach, Stuttgart (DE); Tjalf Pirk, Stuttgart (DE)

(21) Appl. No.: 18/170,370

(22) Filed: Feb. 16, 2023

Related U.S. Application Data
(60) Provisional application No. 63/268,807, filed on Mar. 3, 2022.

(51) Int. Cl. A61M 60/178; A61M 60/216; A61M 60/438; A61M 60/508; A61M 60/818

(52) U.S. Cl. CPC A61M 60/178; A61M 60/216; A61M 60/438; A61M 60/508; A61M 60/818; A61M 2205/3331

(57) ABSTRACT

A sensor device for sensing at least one functional value of a medical device is described herein. The sensor device includes a micro-electronic-mechanical system, an attachment device, and a communication interface. The micro-electronic-mechanical system senses (for example, determines or detects) a functional value associated with a medical device. The attachment device attaches the sensor device to a medical device, a part of the body of a patient, or to the patient intracorporeally. The communication interface provides (for example, wirelessly transmits) at least one functional value to an external device.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

The invention disclosed herein is generally related to systems and methods for sensing at least one functional value of a medical device and providing improved periodic and regular monitoring of the medical device. U.S. Pat. No. 4,889,131 generally describes a portable belt monitor for physiological functions and sensors for the portable belt monitor.

### SUMMARY

The sensor device for sensing at least one function value of a medical device can include a micro-electronic-mechanical system, an attachment device, and a communication interface. The micro-electronic-mechanical system can determine a functional value from a medical device. The attachment device can attach the sensor device to a medical device, a human body part and/or intracorporeally. The communication interface can provide at least one functional value to an external device.

The medical device may be an implantable medical device. For example, the medical device may be a pump unit that is part of a cardiac support system. The functional value can give an indication of proper operation of the medical device (for example, proper operation of a pump unit). The micro-electronic-mechanical system (MEMS) can be a miniaturized assembly, which can include logic elements and/or micromechanical structures, such as sensors. The micromechanical structures may be combined in one chip. The size of a sensor of the MEMS can be in the range of a few micrometers to less than one micrometer. The MEMS can be cheaply mass-produced. To attach the sensor device to the medical device, the attachment device can, for example, be shaped to connect (for example, via a friction-fit) to the medical device. In some examples, the attachment device may be a belt or a plaster that can be attached to a patient's body part (for example, torso, arm, leg, and so forth). To attach the sensor device intracorporeally, the attachment device and/or the entire sensor device may be shaped appropriately for implantation (for example, in a canal). The communication interface can operate via wireless communication to transmit, for example, a functional value through human tissue to an externally located medical facility or an external device. This allows for efficient monitoring of medical device operation. For example, a sensor device implanted intracorporeally can be coupled with a medical device to enable continuous monitoring of the functional values of the medical device, enabling a safe mechanism to monitor medical device functionality. In another example, a sensor device attached to a human body part can allow a patient to monitor functional values as often as required. This can reduce the length and number of medical consultations, by allowing the patient to perform continuous or periodic self-monitoring.

The communication interface can be a Bluetooth interface and/or a charging interface for fast, automatic, local transmissions of the functional value to an external device.

The MEMS may include a single sensor, multiple sensors of a single type, or a combination of sensors of various types for detecting functional values. Examples include a microphone for detecting an acoustic functional value, a structure-borne sound sensor for detecting an oscillation and/or vibration functional value, rotation rate sensors for detecting macroscopic movement as a functional value, magnetic sensor devices for detecting body alignment as a functional value, chemical sensor devices for detecting body function as a functional value, and optical sensor devices for detecting an optical functional value. The MEMS can be useful when performing auscultation or listening to medical devices. For example, sensors can be used to detect a blockage or imminent blockage of a pump unit of a medical device. In this example, a structure-borne sound sensor can be used to detect vibration and/or oscillation of the human body, acceleration and/or rotation rate sensor can be used to detect a macroscopic body movement, or the magnetic sensor device can be used to detect alignment (or misalignment) of the human body.

In some examples, a structure-borne sound sensor can include an ultrasonic sensor device.

The sensor device may include a power supply device for autonomous operation of the MEMS. The power supply device may, for example, include an energy storage device with at least one battery, and/or a charging device for providing energy to the energy storage device. This can extend the operation time of the sensor device.

The power supply device can allow charging of an energy storage device by, for example, utilizing kinetic energy or via an inductive charging interface. For example, the power supply device can provide power for the energy storage by using an “Energy-Harvester” that can detect movements of a user (for example, a patient) and convert the kinetic energy associated with the movement of the user to an electrical energy. This allows for a sensor device that is self-sufficiently operable. An inductive charging interface may also charge the energy storage device.

In some examples, the sensor device may include a control electronics unit for energy control, data control, data processing and/or communication control of the sensor device.

The sensor device may have an evaluation device that can compare a functional value of a medical device with a stored target value. Additionally, the evaluation device may provide a comparison result. The evaluation device can provide a positive comparison result if the functional value corresponds to the target functional value. Additionally and/or alternatively, the evaluation device can provide a negative comparison result if the functional value differs from (for example, less than or greater than) the target functional value. The evaluation device can transmit a comparison result to an external device, for example to a medical device, smartphone, smartwatch, and/or directly to hospital software via the communication interface described herein. This can allow quick and direct evaluation of the functional value by the patient and/or care providers. In some examples, the sensor device can generate a warning signal indicating that the medical device is not functioning properly based at least in part on negative comparison results.

The sensor device may include an auxiliary device separate from the sensor device that can communicate with the sensor device and/or to control the sensor device. The auxiliary device may include at least one counter-sensor (for example, for the MEMS), and an external auxiliary equipment. This allows for a communication, control, and/or sensing process to be carried out in a user-friendly manner by the patient by using the external auxiliary equipment of the auxiliary device.

The external auxiliary equipment may include, for example, an operating device for operating the sensor device and/or a transmitting device for providing data to the sensor device. For example, data may be remotely transmitted using General Packet Radio Service (GPRS).

The sensor device may include a memory device that can store at least one target functional value for comparison with a functional value (for example, detected or determined by the sensor device described herein). This allows the target functional value to be readily available for comparison. The target functional value can include, for example, sound fragments and/or fingerprints, such as samples or wavelets. Sensed (for example, detected or determined) functional value signals can be compared to the target functional value to determine the condition of a medical device. The target functional value can be, for example, a predefined target functional value, or a target functional value adjusted over the operating time of a medical device. In some examples, the target functional value may be user-configurable.

In some examples, the sensor device can be attached to a coupling device of a cardiac support system. A cardiac support system can include a coupling device including a pump unit. By attaching a sensor device to the coupling device of a cardiac support system, a patient may be able to self-monitor components or operation of the cardiac support system.

According to one aspect, a method of operating a sensor device is disclosed. The method can include a sensing step and a providing step. In the sensing step, a functional value is sensed (for example, detected or determined) when the sensor device is attached to a medical device, a human body part, or intracorporeally. In the providing step, at least one functional value is provided to an external device. The method can be implemented in software or hardware, or in a mixture of software and hardware. The software and/or hardware may be a part of a control unit, for example. Prior to the sensing step, the method can include an additional step in which the attachment device of the sensor device is attached to a medical device, a human body part and/or intracorporeally.

A computer program product and/or computer program with program code may perform and/or implement and/or control at least one of the steps of the method described above. The computer program product and/or computer program with program code may be stored on a machine-readable carrier or storage medium, such as a semiconductor memory, a hard disk memory, or an optical memory.

### BRIEF DESCRIPTION OF THE DRAWINGS

FIG. 1A is a schematic diagram showing an embodiment of a MCS system with a monitoring device.

FIG. 1B is a cross-sectional view of a heart showing an example catheter and positioned across an aortic valve via a femoral artery access.

FIG. 2 schematically illustrates an access pathway for a catheter.

FIG. 3 is a side view of an example catheter for delivering an example mechanical circulatory support (MCS) device.

FIG. 4 illustrates the catheter of FIG. 3, with an introducer sheath removed.

FIG. 5 is a cross-sectional view of an impeller region of the MCS device of FIG. 3.

FIG. 6 is a system diagram of an example sensor device for sensing at least one functional value of a medical device.

FIG. 7 is a system diagram of another example of a sensor device.

FIG. 8 is a schematic illustration of an example implementation of a sensor device.

FIG. 9 a schematic diagram of an example cardiac support system including a pump unit and a sensor device.

FIG. 10 is a flow chart illustrating an example method of operating a sensor device.

### DETAILED DESCRIPTION

In some embodiments a sensor system 100 may be part of or used in conjunction with an implantable medical device 110, which may be a cardiac support system. A cardiac support system may include a mechanical circulatory support (MCS) device that may include a temporary (for example, generally no more than about 6 hours or no more than about 3 hours, 4 hours, 5 hours, 7 hours, 8 hours, or 9 hours) left ventricular support device for use during a high-risk percutaneous coronary intervention (PCI) performed for elective or urgent, hemodynamically stable patients with severe coronary artery disease and/or depressed left ventricular ejection fraction. The system may be used when a heart team, including a cardiac surgeon, has determined high risk PCI is the appropriate therapeutic option. For example, the MCS system is placed across the aortic valve, for example via a single femoral arterial access. In some embodiments, the MCS device may be a heart pump, a ventricular assist device (VAD), a left ventricular assist device (LVAD), mechanical circulatory support device, a mechanical cardiovascular assistance device, or other medical device that can provide long-term cardiac support (for example, more than 30 days). Any of the sensor devices or systems described herein, or features thereof, may be used in any of the heart pump devices or systems described in U.S. Patent Publication No. 2022/0161019, filed Nov. 18, 2021, titled PURGELESS MECHANICAL CIRCULATORY SUPPORT SYSTEM WITH MAGNETIC DRIVE; in U.S. Patent Publication No. 2022/0161018, filed Nov. 18, 2021, titled MECHANICAL CIRCULATORY SUPPORT SYSTEM WITH GUIDEWIRE AID; and/or in U.S. Patent Publication No. 2022/0161021, filed Nov. 18, 2021, titled MECHANICAL CIRCULATORY SUPPORT SYSTEM WITH INSERTION TOOL, the entire disclosure of each of which is incorporated herein by reference and forms a part of this specification for all purposes.

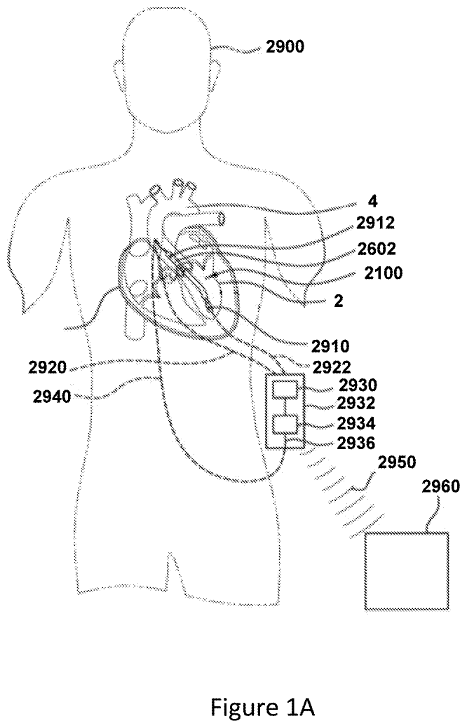

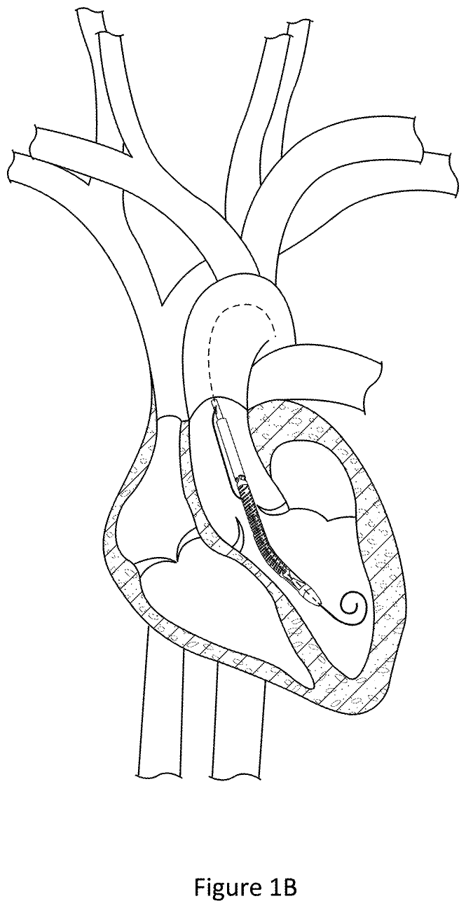

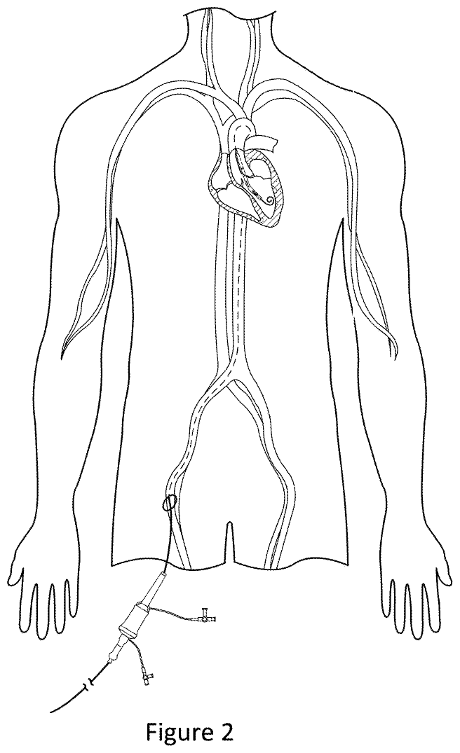

FIG. 1A shows another embodiment of an MCS system 10 implanted in a patient 2900. The MCS system 10 may be used with any of the systems, devices, or methods described with respect to FIGS. 1B-10. As shown in FIG. 1A, the MCS system 10 may interact with a monitoring device 2932 for monitoring a state of health of the patient 2900. The MCS system 10 may include an MCS device 2100, such as the pump 22 described herein, which can pump blood from the ventricle 2 of the heart 1 into the aorta 4 of the patient 2900. The MCS device 2100 may include a first pressure sensor 2910 and a second pressure sensor 2912. The first pressure sensor 2910 may generate and send a first pressure signal 2920 to the monitoring device 2932. The second pressure sensor 2912 can send a second pressure signal 2922 to the monitoring device 2932. The pressure signals 2920, 2922 may be transmitted to the monitoring device 2932 wirelessly or via a wire or a cable.

The first pressure sensor 2910 and the second pressure sensor 2912 may be positioned at a predetermined distance from one another in the MCS device 2100, so that they can detect, for example, the blood pressure, blood pressure fluctuations or a pulse wave of blood. The monitoring device 2932 may include a reading interface (or input/output interface) 2930 which may receive the first pressure signal 2920 and the second pressure signal 2922. The pressure signals 2920, 2922, once received, may be forwarded to a processing unit 2934, which may then determine a processing value 2936. The processing value 2936 may then be used to determine a state of health of the patient 2900. By monitoring and tracking the processing values 2936, the state of health of the patient 2900 may be monitored.

In some embodiments, the processing value 2936 may be a transit time of the pulse wave of blood between the first pressure sensor 2910 and the second pressure sensor 2912. Alternatively or additionally, such a processing value 2936 may also represent a parameter that represents an elasticity of vessel walls, such as a wall of the aorta 4. As such, the processing value 2936, for example, may be used to determine the patient's state of health with regard to the elasticity of vessel walls. For example, the processing value 2936 may be used to determine, characterize, or estimate the amount of deposits or calcifications on the inner walls of the vessels.

The monitoring device 2932 may generate a control signal 2940 based on the processing value 2936. For example, the control signal 2940 may control the MCS device 2100 of the MCS system 10 in order to provide sufficient amount of blood flow or generate an artificial increase in blood pressure to allow the patient 2900 to participate in a desired or specific activity (for example, climbing stairs).

The monitoring device 2932 may generate and transmit (wirelessly or via a wire) a signal 2950 to a separate computer unit 2960 (for example, a data server such as a cloud server) based on the processing value 2936, the pressure signals 2920, 2922, and/or the control signal 2940. The signal 2950 may include a notification or an evaluation related to the pressure signals 2920, 2922, the processing value 2936, and/or the control signal 2940. In some embodiments, the monitoring device 2932 may be worn externally to the patient 2900. For example, the monitoring device 2932 may be attached to a belt of the patient 2900. Alternatively, the monitoring device 2932 may be an integral component of the MCS system 10, such that the pressure signals 2920, 2922 may be transmitted to the monitoring device 2932 via wires or cables. If the monitoring device is implanted in the patient 2900, the monitoring device 2932 may include an energy storage device (for example, a long-life, rechargeable battery). The energy storage device (not shown) of the monitoring device 2932 may be charged via a power, supplying cable or a wireless power transmission system.

In some embodiments, the monitoring device 2932 may be divided into multiple components. For example, the reading interface 2930 may be implanted in the patient 2900 while the processing unit 2934 may be positioned externally (for example, worn on a belt of the patient 2900), where the reading interface 2930 and the processing unit 2934 may wirelessly communicate with one another.

In some embodiments, one of the pressure sensors 2910, 2912 may be arranged outside the patient 2900. For example, one of the pressure sensors 2910, 2912 may be placed in the monitoring device 2932. The pressure value obtained from the other pressure sensor positioned inside the patient 2900, which may then represent the patient's blood pressure, can be normalized. This allows calculations of the absolute blood pressure value of the patient 2900 can be done reliably while allowing compensation for any systematic errors (such as a change in the ambient air pressure around the patient 2900, for example when changing floors in a house, weather-based changes in air pressure or topographic altitude). This allows the patient's state of health to be determined very reliably in different environmental scenarios.

Additional features that may be included in the embodiment of the MCS system 2100, and in any related components and/or features described or shown in FIG. 1A or elsewhere herein, include those described in PCT Publication No. WO2020030706, filed Aug. 7, 2019, titled DEVICE AND METHOD FOR MONITORING THE STATE OF HEALTH OF A PATIENT, which is hereby incorporated by reference herein in its entirety and forms a part of this specification for all purposes.

The MCS system 10 can include a low-profile axial rotary blood pump mounted on a catheter (for example, an 8 French (Fr) catheter). The pump may be referred to as an MCS pump or MCS device. When in place, the pump can be driven by an MCS controller to provide up to about 4.0 liters/minute of partial left ventricular support, at about 60 mm Hg. The MCS pump can include an improved bearing and/or a sealed motor that can reduce the need (or prevent) for system purging. In some examples, the MCS system is visualized fluoroscopically such that it can be positioned at a desired location without any sensors.

The MCS system 10 can include an expandable sheath that can allow 8-10 Fr initial access size for easy insertion and closing, and expandable to allow introduction of 14 Fr and 18 Fr pump devices and return to a narrower diameter around the 8 Fr catheter once the pump has passed. For example, the expandable sheath can allow passage of the heart pump through vasculature while minimizing shear force within the blood vessel, reducing the risk of bleeding and healing complications. Distention or stretching of an arteriotomy may be done with radial stretching with minimal shear, which is less harmful to the vessel. Access may be accomplished via transfemoral, transaxillary, transaortal, or transapical approach.

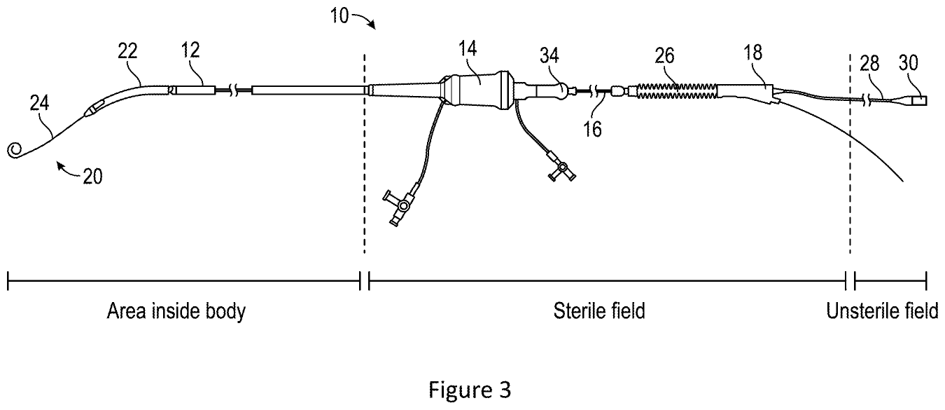

FIG. 1B illustrates a cross-sectional view of a heart showing a rendering of the MCS pump 22 mounted on the tip of an 8 Fr catheter 16. As used herein, “distal” and “proximal” refer to directions along the MCS system 10 that are, respectively, toward the end that is inside the body, and toward the end that remains out of the body or toward the unsterile field, as further shown in FIG. 3. The pump can include an inlet tube portion 70 that extends across the aortic valve. The pump can include an impeller located at an outflow section of the inlet tube. The impeller can draw blood from the left ventricle and ejecting the drawn blood into the ascending aorta. The pump can include a motor mounted directly proximal to the impeller in a housing. In some example, the housing can be sealed to eliminate the need to flush the motor prior to or during use. This configuration provides hemodynamic support during high-risk PCI, and time and safety for a complete revascularization via a minimally invasive approach (rather than an open surgical procedure).

The MCS system 10 can eliminate (or reduce) the need for motor flushing, and provide increased flow performance up to 4.0 L/min at 60 mmHg with acceptably safe hemolysis by using a computational fluid dynamics (CFD) optimized impeller that minimizes shear stress.

The MCS pump 20 can actively unload the left ventricle by pumping blood from the ventricle into the ascending aorta, thereby providing systemic circulations. When in place, the MCS pump can be driven by a MCS Controller to provide between 0.4 L/min up to 4.0 L/min of partial left ventricular support.

The MCS system can include combinations of one or more of the following features:
A pump including a shaft, a proximal hub, an insertion tool, a proximal cable, an infection shield, and guidewire aid. The MCS Device is provided sterile.
The shaft containing electrical cables and a guidewire lumen for over-the-wire insertion.
The proximal hub containing a guidewire outlet with a valve to maintain hemostasis. The proximal hub can connect to the shaft to the proximal cable. that connects the pump to a MCS controller.
The proximal cable is 3.5 m (approx. 177 inch) in length and extends from a sterile field of the MSC system to a non-sterile field of the MSC system (for example, where a MCS controller is located).
A MCS insertion tool as part of the MCS Device to facilitate the insertion of the pump into the Introducer Sheath and to protect the inlet tube and the valves from potential damage or interference when passing through the Introducer Sheath.
A peel-away guidewire aid pre-mounted on the MCS Device to facilitate the insertion of the 0.018″ placement guidewire into the pump and into the MCS shaft.
A 3 m 0.018″ placement guidewire, having a soft coiled pre-shaped tip for atraumatic wire placement into the left ventricle. The guidewire is provided sterile.
A 14 Fr Introducer Sheath with a usable length of 275 mm to maintain access into the femoral artery and provide hemostasis for the 0.035″ guidewire, the diagnostic catheters, the 0.018″ placement guidewire, and the insertion tool. The housing of the Introducer Sheath is designed to accommodate the MCS Insertion Tool. The Introducer Sheath is provided sterile.
An introducer dilator compatible with the Introducer Sheath to facilitate atraumatic insertion of the Introducer Sheath into the femoral artery. The introducer dilator is provided sterile.
An MCS Controller which drives and operates the MCS Device, observes its performance and condition as well as providing error and status information. The powered controller is designed to support at least about 12 hours of continuous operation and contains a basic interface to indicate and adjust the level of support provided to the patient. Moreover, the controller provides an optical and audible alarm notification in case the system detects an error during operation. The MCS Controller is provided non-sterile and is contained in an enclosure designed for cleaning and re-use outside of the sterile field. The controller enclosure contains a socket into which the extension cable is plugged.

FIG. 3 illustrates an example MCS system 10 in accordance with one aspect of the present disclosure. The MCS system 10 can include an introducer sheath 12 having a proximal introducer hub 14 with a central lumen for axially movably receiving a shaft 16. The introducer hub 14 can include a proximal hub 34. The shaft 16 can extend between a proximal hub 18 and a distal portion 20. The hub 18 may be provided with an integrated microcontroller or memory storage device for device identification and tracking of operation time (for example, total operation time), which could be used to prevent overuse to excessive wear (or other technical malfunction). The microcontroller or memory device could disable the MCS system, for example, to prevent using a used component (for example, a used pump or a shaft). The microcontroller or memory device of the MCS system can communicate with a MCS controller to display information about the MCS system or messages about usage of the MCS system.

In some implementations, the pump can include an atraumatic cannula tip with radiopaque material that can allow the MCS system (for example, the pump) to be visible using fluoroscopy during implantation or explantation.

The MCS system can include a pump 22 coupled to a distal region of the shaft 16. The MCS system 10 (for example, the pump 22, the introducer hub 14, and the proximal hub 18 of the MCS system 10) can include at least one central lumen for axially movably receiving a guide wire 24. The proximal hub 18 can include an infection shield 26. The MCS system 10 can include a proximal cable 28 that can extend between the proximal hub 18 and a connector 30. The connector 30 of the MCS system 10 can provide removable connection between the MCS system 100 and a control system (for example, typically outside of the sterile field) to, for example, receive electronic signals that can drive the pump 22.

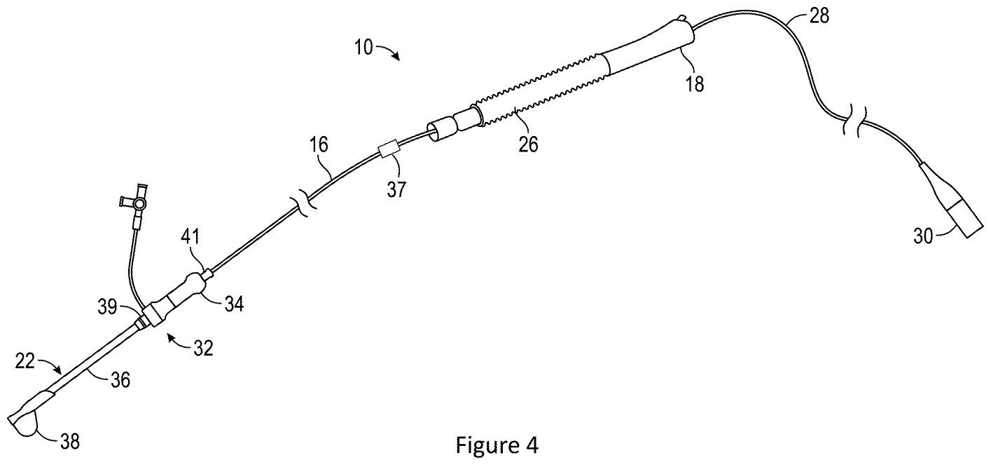

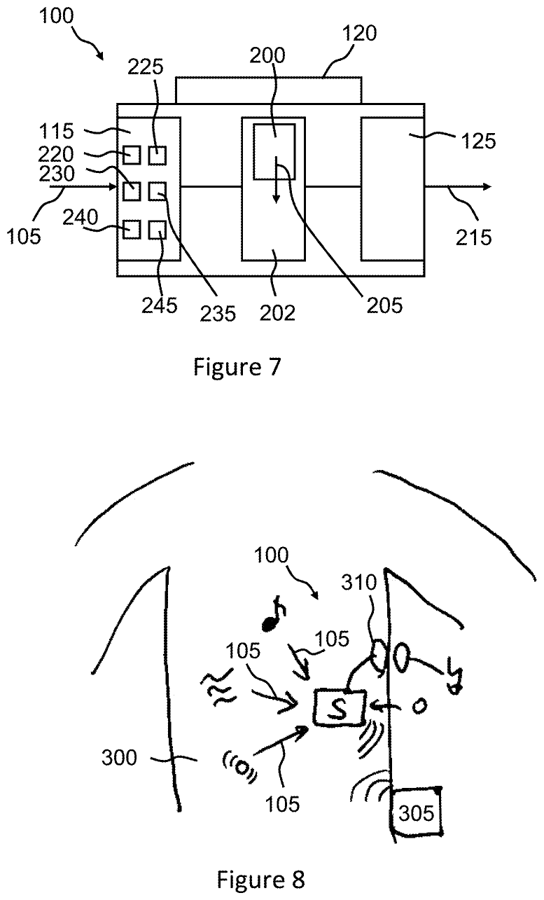

FIG. 4 illustrates the MCS system 10 with the introducer sheath 12 removed, showing an insertion tool 32. The insertion tool 32 can have an elongate tubular body 36 having a length between about 85 mm and about 160 mm (e.g., about 114 mm), and an inside diameter between about 4.5 mm and about 6.5 mm (e.g., about 5.55 mm), and extending distally from the proximal hub 34. The tubular body 36 can include a central lumen that can axially movably receive the MCS shaft 16 and pump 22 therethrough, and have sufficient collapse resistance to maintain patency when passed through the hemostatic valves of the introducer sheath 12. As illustrated in FIG. 4, the pump 22 can be the elongate tubular body 36 so as to facilitate passage of the pump 22 through the hemostatic valve(s) on the proximal end (for example, about the proximal hub 34) of an introducer hub 14. In some examples, a marker 37 is provided on the shaft 16 spaced proximally (for example, at a predetermined distance) from the distal tip 64 (see FIG. 7) such that as long as the marker 37 is visible on the proximal side of the proximal hub 34, a care provider (for example, a physician) knows that the pump 22 is within the tubular body 36.

The proximal hub 34 can include a first engagement structure 39 that can engage a corresponding second engagement structure on the introducer sheath 12 to couple the insertion tool 32 with the introducer sheath 12. Additionally, the proximal hub 34 can include a locking mechanism 41 that can attach (for example, removably attach) to the shaft 16 to prevent the shaft 16 from sliding proximally or distally through the insertion tool 32 once, for example, the pump 22 is positioned at a desired location in the heart. Additionally, the proximal hub 34 can include a hemostasis valve to seal around the shaft 16 and accommodate passage of a pump with a larger diameter. In one commercial presentation of the MCS system 10, the pump 22 can be packaged such that it is pre-positioned within the insertion tool 32 and a guidewire aid 38 is pre-loaded within the pump 22 and the shaft 16, as illustrated in FIG. 4.

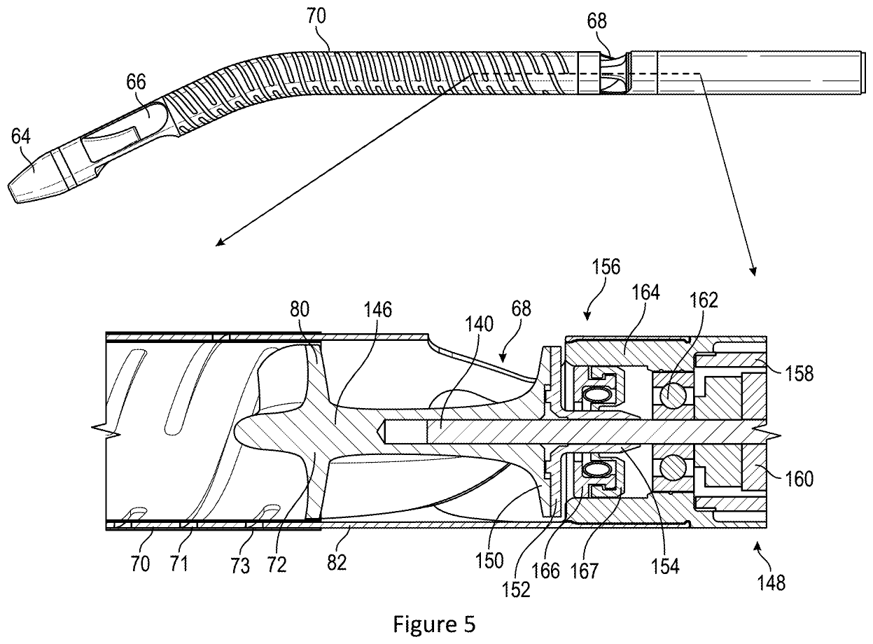

FIG. 5 illustrates a side view of the pump 22 and a partial cross-sectional view of the pump 22. As shown in FIG. 5, the impeller 72 can be attached to a motor drive shaft 140. The motor drive shaft 140 can be rigid. In the example shown in FIG. 5, the drive shaft 140 can extend distally into a proximally facing central lumen 142 of the impeller 72. Additionally, the drive shaft 140 can extend through a sleeve 154 of an impeller back 152 coupled to the impeller hub 146. The sleeve 154 can be tubular in shape. The sleeve 154 may be secured to the impeller hub 146 by a press fit, laser weld, adhesives or other suitable bonding techniques. The impeller 72 can includes a radially outwardly extending helical blade 80, which, at its maximum outside diameter, is spaced apart from the inside surface of an impeller housing 82 between about 40 μm and about 120 μm. The impeller housing 82 can be tubular. The impeller housing 82 may be a proximal extension of inlet tube 70. Additionally, the impeller housing 72 may be positioned on a proximal side of slots 71 formed in the inlet tube 70 to provide flexibility distal to the impeller 72. A tubular outer membrane 73 can enclose the inlet tube 70 and seal the slots 71 while preserving flexibility of the inlet tube 70. Pump outlets 68 can be formed in the sidewall of the impeller housing 82, aligned with at least a portion of the impeller 72. For example, the pump outlets 68 can be axially aligned with at least a portion of the impeller 72 as shown in FIG. 5. The pump outlets 68 can overlap with between about 10% and about 50% of a proximal portion of the impeller 72.

The impeller 72 may be made out of a medical grade titanium. This can enable a computational-fluid-dynamics (CFD) optimized impeller design with minimized shear stress for reduced damage of the blood cells (hemolysis) and a non-constant slope increasing the efficiency. Electro polishing of the surface of the impeller 72 can decrease the surface roughness to minimize the impact on hemolysis.

In one implementation of the invention, the impeller hub 146 can flare radially outwardly in a proximal direction to form an impeller base 150. The impeller base 150 can direct blood flow out of the outlets 68. A proximal surface of the impeller base 150 can be secured to an impeller back 152, which may be in the form of a radially outwardly extending flange, secured to the motor shaft 140. The sleeve 154 of the impeller back 152 may form a central aperture to receive the motor shaft 140. Additionally, the impeller back 152 may be integrally formed with or bonded to the sleeve 154 that can be coupled to (for example, bonded to) the motor shaft 140. In one implementation, the impeller back 152 can be first coupled to the motor shaft 140 and bonded such as through the use of an adhesive. The impeller 72 may then be advanced over the shaft 140 and the impeller base 150 coupled (for example, bonded by welding) to the impeller back 152.

The opening of the sleeve 154 may increase in inner diameter in a distal direction (for example, towards the impeller 72), to facilitate application of an adhesive. The proximal end of the sleeve 154 may decrease in outer diameter in a proximal direction (for example, away from the impeller 72) to form an entrance ramp that can facilitate advancement of the sleeve 154 in a proximal direction (for example, away from the impeller 72) over the motor shaft 140 and through the motor seal 156, discussed further below.

A motor 148 can includes a stator 158 having conductive windings surrounding a cavity enclosing a motor armature 160. The motor armature 160 can include a plurality of magnets that, for example, can be rotationally secured with respect to motor shaft 140. The motor shaft 140 can extend from the motor 148 through a rotational bearing 162 before exiting the sealed motor housing 164. Additionally, the motor shaft 140 can extend through a seal 156. The seal 156 can include a seal holder 166 which supports a seal body 167. The seal body 157 can be annular, such as a polymeric seal ring. The seal body 167 can include a central aperture for receiving the sleeve 154. Additionally, the seal body 167 can be biased radially inwardly against the sleeve 154 to facilitate (for example, maintain) a contact between the seal ring 167 and the sleeve 154. In some example, the outside surface of the sleeve 154 may be provided with a smooth surface such as by electro polishing, to minimize wear on the seal 156.

The pump (for example, the pump 22) can include a sealed motor (for example, the motor 148) due to the short time of usage for high risk PCI (typically no more than about 6 hours), configured for use without flushing or purging. This configuration can provide the opportunity to directly bond the impeller (for example, the impeller 72) on the motor shaft (for example, the shaft 140) as discussed described herein, removing issues that can be associated with magnetic coupling such as the additional stiff length, space requirements, or pump efficiency. In some example, a four pole motor configuration may be used. The four pole motor configuration can enable flow performance up to 4.0 L/min at 60 mmHg with low temperature change. The motor cable interface can be provided with a high tensile strength.

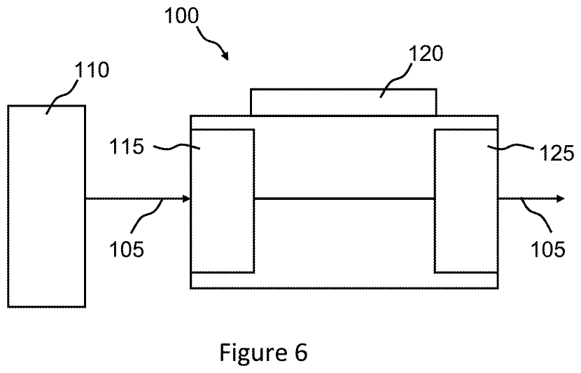

FIG. 6 shows a schematic representation of an example sensor device 100 for sensing (for example, detecting, measuring, or determining) at least one functional value 105 of a medical device 110. The medical device 110 may be a long-term cardiac support device such as a heart pump, a ventricular assist device (VAD), a left ventricular assist device (LVAD), mechanical circulatory support device, a mechanical cardiovascular assistance device, or other medical device.

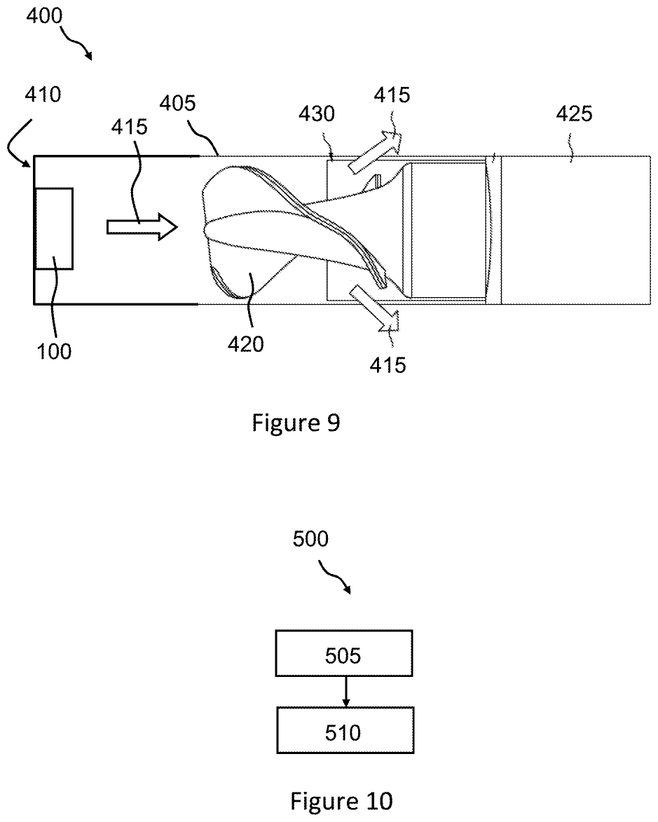

The sensor device 100 can include a micro-electronic-mechanical system (MEMS) 115, an attachment device 120, and a communication interface 125. The MEMS 115 can sense (for example, detect, measure, or determine) a functional value 105. The attachment device 120 can attach the sensor device 100 to the medical device 110 or to a body of a patient (for example, a portion of the body or intracorporeally). The communication interface 125 can transmit at least one functional value 105 to an external device, or to a remote system (for example, a remote server or an extracorporeal device). The medical device 110 may or may not be implantable. For example, the medical device 110 may be a pump unit 405 of a cardiac support system 400 (see FIG. 9). The functional value 105 may, for example, provide an indication of the operation status of the medical device 110.

In some implementations, the MEMS 115 can be a miniaturized assembly including logic elements or micromechanical structures (for example, sensors), which may be combined in one chip. In some examples, the sensors of the MEMS 115 can range in size from a few micrometers to less than one micrometer. To attach the sensor device 100 intracorporeally, the attachment device 120 or the sensor device 100 can be shaped or dimensioned to be suitable for implantation. For example, the attachment device 120 or the sensor device 100 can be shaped to be suitable for implantation in a canal, a heart, a blood vessel, an intracorporeal space.

In some implementations, the attachment device 120 can allow the sensor device 100 to be attached extracorporeally to a body of a patient. For example, the attachment device 120 can be a belt or plaster.

The communication interface 125 can be a wireless communication interface that can establish wireless communication with another device (for example, a remote computing or monitoring device) and wirelessly transmit the functional value 105 (for example, through the tissue of the patient).

The sensor device 100 described herein can also be referred to as a “system for intracorporeal auscultation.” Auscultation refers to listening to the sounds of the human body, for example the sounds of the heart or lungs. This is typically done with a stethoscope, through which sound phenomena are transported. Acoustic and electronic stethoscopes capture sound phenomena using different methods. Acoustic stethoscopes have a membrane that transmits acoustic waves to an air column in the stethoscope tube. On the other hand, electronic stethoscopes can electronically pick up and amplify sound, and can highlight or mask various sounds. The auscultation may be a part of a physical examination conducted by trained doctors. During auscultation, various areas of the body can be listened to, for example the lungs, heart, abdomen, and/or blood vessels. By listening to these sounds with a trained human ear, a doctor can make assessments about the condition of a patient's body, for example, damage in the heart and/or heart valves, or defects in the cardiac septum.

The sensor device 100 described herein can allow continuous or periodic auscultation that can be performed by a care provider (for example, a doctor) or by a patient.

When devices (for example, the medical device 110) are operating inside a patient body (for example, permanently or temporarily), monitoring of such devices can be challenging. One example of such device operating inside a patient's body is a mechanical cardiovascular assistance device, or as known as “Ventricular Assist Device,” (VAD). A VAD can include fast rotating parts and bearings. A major problem associated with the use of a VAD is the potential proliferation of endothelial cells. These endothelial cells can form at and detach from an inlet wall of a VAD and flow into the blood stream as, for example, a pannus. These cells can travel in the bloodstream until they reach a narrow site of a blood vessel and can cause occlusion, which can lead to strokes in VAD patients. Early detection or prediction of potential complications can prevent issues such as proliferation of endothelial cells but it usually requires an operation.

The sensor device 100 can provide care providers (for example, physicians) or patients the ability to detect potential issues with their medical devices (for example, permanently or temporarily implanted) early via auscultation. This can be important in situations that require more frequent monitoring of signs of issues (for example, proliferation of endothelial cells), such as with critical patients and patients with implanted medical devices 110.

For patients with critical conditions or with implanted medical devices, it may be advantageous to perform auscultation outside of medical facilities (for example, a hospital or a doctor's office). The sensor device 100 described herein allows for auscultation outside of medical facilities (for example, at home), and can be used for a wide range of instruments (for example, micro-implants).

The sensor device 100 can be implanted (for example, permanently or temporarily) in the body in a previously defined (for example, predetermined) region in order to perform an auscultation on-site at a medical facility. Auscultation can also be performed remotely away from a medical facility, and the data of the auscultation can be wirelessly transferred to an external device or a server associated with a care provider or a care provider facility.

In some implementations, the sensor device 100 can be positioned remotely from an implanted medical device (for example, the medical device 110). For example, the sensor device can be attached to a belt or placed in a patch that can wrap around a part of a patient's body. Such an extracorporeal sensor device 100 can be worn regularly or periodically, and can collect, for example, functional value from the implanted medical device regardless of whether the patient is in motion. In some examples, the sensor device 100 (for example, extracorporeal sensor device) can be attached to a patient using an attachment system (for example, a belt) with a microphone positioned at a location determined by a qualified personnel (for example, a physician, a nurse, or a care provider). Thus, home measurement (for example, of auscultation data) is possible with appropriate patient training. The sensor device 100 can be removably attached to the body of a patient using the attachment device 120 for long-term- or short-term application.

FIG. 7 illustrates a schematic representation of an example operation of the sensor device 100, with the difference that the sensor device 100 in FIG. 7 includes a storage device 200 and/or an evaluation device 202.

As shown in FIG. 7, at least one target functional value 205 is stored in the storage device 200 for comparison with the functional value 105. For example, the target functional value 205 is a pre-defined (or predetermined) target functional value 205. Optionally, the target functional value 205 can be adjusted and applied over a period of time (for example, an operating time of a medical device 110).

The evaluation device 202 can compare the functional value 105 with the stored target functional value 205. Additionally, the evaluation device 202 can provide a comparison result 215 (for example, of the functional value 105 and the target functional value 205). The evaluation device 202 can provide a positive comparison result if the functional value 105 corresponds to the target functional value 205. Alternatively, the evaluation device 202 can provide a negative comparison result if the functional value 105 differs from the target functional value 205. The evaluation device 202 can, via the communication interface 125, provide (for example, wirelessly transmit) the comparison result 215 to an external device, such as a medical device, smartphone, smartwatch, or a care provider server. A negative comparison result may be transferred from the evaluation device 202, via the communication interface 125, to an external device to provide a warning signal indicating the medical device is not functioning as expected. In some examples, a negative comparison result can include a warning signal.

In some implementations, the evaluation device 202 can receive the target functional value 205 from a storage unit or a system external of the sensor device 100. For example, the target functional value 205 may be transmitted wirelessly to the evaluation device 202.

The sensor device 100 can include a control electronics unit (or a controller) that can control power distribution, data transmission, data processing, communication (for example, wireless or wired) for the sensor device 100. The evaluation device 202 may be implemented as a part of the control electronics unit.

The sensor device 100 can be an implantable device using the MEMS 115 with one or more integrated sensors. For example, the MEMS 115 can include at least one microphone 220 for detecting acoustic data as the functional value 105, a structure-borne sound sensor 225 for detecting a vibration or oscillation data as the functional value 105, an acceleration and/or rotation rate sensor 230 for detecting a macroscopic movement data as the functional value 105, a magnetic sensor device 235 for detecting an orientation data as the functional value 105, a chemical sensor device 240 for recording a body function data as the functional value 105 and/or an optical sensor device 245 for recording an optical data as the functional value 105, and the like.

One or more of the sensors 220, 225, 230, 235, 240, 245 can be used to detect, for example, a blockage or imminent blockage of a pump unit of an implanted (for example, permanently or temporarily) medical device. For example, the structure-borne sound sensor 225 may detect vibration or oscillation of the body or a portion of the body of a patient, the acceleration and/or rotation rate sensor 230 may detect a macroscopic movement of the body or a portion of the body of the patient, and the magnetic sensor device 235 may detect an alignment of the body or a portion of the body of the patient. The structure-borne sound sensor 225 can include an ultrasonic sensor device.

The MEMS 115 can include one or more of the following, but not limited, sensors:
A structure-borne sound sensor for detection of oscillations and/or vibrations in the body (can include an ultrasonic sensor device for monitoring local areas of the body)
A microphone for recording acoustic signals
An acceleration and/or rotation rate sensor for macroscopic movement of the patient (for example, running)
A magnetic sensor for determining body alignment and detecting magnetically marked particles introduced to the body.
A chemical sensor for monitoring body functions
An optical sensor for measuring optical parameters

The sensor device 100 may be either implanted directly into the affected body region with an attachment device 120, or may be incorporated into an implanted device, for example a VAD.

Vibrations may be converted into an electrical signal which can in turn can be converted into a noise. The sensor device 100 can function autonomously while implanted in the body of a patient, with no external power source. For example, the sensor device 100 can be equipped with a power supply device for autonomous operation of the MEMS 115. The power supply device can include an energy storage device with at least one battery and a charging device. The energy storage device may include an “Energy-Harvester” described herein that can convert the patient's kinetic energy into an electrical energy, which is then stored in the energy storage device. Additionally or alternatively, the charging device may include an inductive charging interface and the energy storage device may receive power from another device via the inductive charging interface.

The communication interface 125 can establish communications (for example, wireless or wired) with other devices as described herein. For example, the communication interface 125 can be a Bluetooth® interface that can establish Bluetooth® based wireless communication with other devices.

With reference to FIG. 8, the sensor device 100 can include an auxiliary device 305 external to the sensor device 100, for example in the form of a supplementary external box. The auxiliary device 305 may add one or more, or all of the functions listed below:
User interface for operation and interaction
General packet radio service (GPRS) or similar methods for remote transmission
Counter sensors for the implanted sensor device (for example, the MEMS 115)

The storage device 200 may be a part of the auxiliary device 305 or an external storage device (for example, a database). Sound fragments and/or fingerprints (such as samples and/or wavelets) may be stored as the target functional values 205, to which functional values 105 can be compared to generate comparison results. Alternatively, the functional values 105 may be transmitted from the communication interface 125 to, for example, a computing device or a server for a medical facility for evaluation.

The auxiliary device 305 can communicate with and control the sensor device 100.

The auxiliary device 305 can include an operating device (for example, a controller or a processor) that can generate electronic signals for controlling operation of the sensor device 100. Additionally, the auxiliary device 305 can include a transmitting device for transmitting the electronic signals (for example, wirelessly) to the sensor device 100 (for example, for controlling operation of the sensor device 100). The transmitting device of the auxiliary device 305 can use various types of suitable data transmission methods including, but not limited to, GPRS, Bluetooth, 3G, 4G, 4G LTE, Zigbee, Wi-Fi, Low-Fi, and the like.

In the example shown in FIG. 8, all components of the sensor device 100 (with the exception of the auxiliary device 305), are placed in the body 300 of a sensor box “S.” The sensor device 100 can receive charge via a charging interface 310. For example, the charging interface 310 can be an inductive charge interface. In some examples, wireless communication can be made via the charging pad 310 (for example, an inductive charging pad). The MEMS 115 housed in the body 300 may sense, among other things, sound, vibrations/oscillations, and chemical reactions. Additionally, the communication interface 125 housed in the body 300 can communicate with other devices such as the auxiliary device 305.

FIG. 9 illustrates a schematic representation of an example of a cardiac support system 400. The cardiac support system 400 can include a pump unit 405, a coupling device, and the sensor device 100 described herein. The cardiac support system 400 can include any of the features or functions as the MCS system 10 described herein, and vice versa.

The pump unit 405 can include a coupling device to which the attachment device 120 of the sensor device 100 is attached. To attach the sensor device 100 to the pump unit 405, the attachment device 120 of the sensor device 100 can be shaped to provide a removable connection with the coupling device of the pump unit 405. The connection between the attachment device 120 and the corresponding coupling device of the pump unit 405 can be a friction-fit or via corresponding locking interfaces (for example, a detent and a corresponding groove).

The sensor device 100 can be attached to an inlet 410 of the pump unit 405, through which fluid 415 (for example, blood) from outside the pump unit 405 is drawn into the pump unit 405. An impeller 420 and a pump drive 425 can facilitate flow through the pump unit 405. In the example illustrated in FIG. 9, the impeller 420 is located between the inlet 410 and the pump drive 425.

During operation of the pump unit 405, the fluid 415 can be drawn into the inlet 410 by rotation of the impeller 420, which is driven by the pump drive 425. The fluid 415 can leave the pump unit 405 through one or more openings 430 (for example, the pump outlets 68) in the impeller casing, arranged radially around the impeller 420. In some examples, the openings 430 can be arranged behind (for example, proximally or away from the inlet 410) the impeller 420.

FIG. 10 shows a flowchart of an example method 500 for operating a sensor device 100. At block 505, the MEMS 115 detects (for example, collects or determines) at least one functional value (for example, the functional values 105). As described herein, the sensor device 100 (which includes the MEMS 115) can be attached to an implanted medical device (for example, the medical device 110), a portion of the body of a patient, or to the patient intracorporeally. At block 510, the sensor device 100 can transmit (for example, wirelessly) the at least one functional value an external device (for example, a server or a computing device of a care provider or a care provider facility).

Optionally, at block 505, the sensor device 100 is attached to a medical device 110, a portion of the body of a patient, or to the patient intracorporeally.

Optionally, the method 500 can be performed recursively.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A system for sensing at least one functional value of a medical device, the system comprising:
a sensor device for sensing at least one functional value of a medical device;
an attachment device configured to couple the sensor device to the medical device; and
a communication interface configured to transmit the at least one functional value to an external device.

2. The system of claim 1, wherein the communication interface is configured to establish Bluetooth® communication with the external device.

3. The system of claim 1, wherein the communication interface comprises a charging interface.

4. The system of claim 1, wherein the sensor device is a micro-electronic-mechanical system (MEMS).

5. The system of claim 4, wherein the MEMS comprises at least one of:
a microphone for collecting acoustic data;
a structure-borne sound sensor for collecting an oscillation and/or vibrational data;
an acceleration and/or rotation rate sensor for generating macroscopic movement data;
a magnetic sensor for collecting alignment as the functional value;
a chemical sensor for collecting body function data as the functional value; or
an optical sensor for detecting optical data as the functional value.

6. The system of claim 1 further comprising a power supply device.

7. The system of claim 6, wherein the power supply device is configured to operate by converting kinetic energy of a user into electrical energy.

8. The system of claim 6, wherein the power supply device is configured to receive energy from an inductive charging interface.

9. The system of claim 1 further comprising an evaluation device configured to compare the functional value with a stored target functional value and to generate a comparison result.

10. The system of claim 1 further comprising an auxiliary device external to the sensor device, wherein the auxiliary device is configured to communicate with and control the sensor device, wherein the auxiliary device comprises at least one counter-sensor for the sensor device.

11. The system of claim 10, wherein the auxiliary device comprises an operating device configure to generate electronic signals for operating the sensor device and a transmitting device configured to transmit the electronic signals to the sensor device.

12. The system of claim 1 further comprising a data storage unit configured to store at least one target functional value for comparison with the functional value.

13. The system of claim 1 further comprising a cardiac support system comprising a coupling device and a pump unit, wherein the sensor device is configured to attach to the cardiac support system via the coupling device.

14. A method for operating a sensor device, the method comprising:
receiving, by a processor of a sensor device, electronic signals from a sensor;
determining, by the processor of the sensor device, at least one functional values associated with a medical device;
retrieving, by the processor of the sensor device, target functional values from a data storage unit of the sensor device;
comparing, by the processor of the sensor device, the at least one functional values with the target functional values; and
transmitting, by the processor of the sensor device via a transmitter device, a comparison result between the at least one functional values and the target functional values to an external device.

15. A non-transitory computer storage unit storing therefore computer-readable instructions that, when executed by a processor of a sensor unit, cause the processor to perform steps of:
receiving, by a processor of a sensor device, electronic signals from a sensor;
determining, by the processor of the sensor device, at least one functional values associated with a medical device;
retrieving, by the processor of the sensor device, target functional values from a data storage unit of the sensor device;
comparing, by the processor of the sensor device, the at least one functional values with the target functional values; and
transmitting, by the processor of the sensor device via a transmitter device, a comparison result between the at least one functional values and the target functional values to an external device.
